(19) United States
(12) Patent Application Publication
(10) Pub. No.: US 2015/0011900 A1
(43) Pub. Date: Jan. 8, 2015

(54) METHOD AND SYSTEM TO FACILITATE BLOOD PRESSURE MANAGEMENT

(71) Applicants: Ying-Chiang Lu, Singapore (SG); Kae Yuan Tan, Singapore (SG); Sundaramoorthy Saravanan, Singapore (SG)

(72) Inventors: Ying-Chiang Lu, Singapore (SG); Kae Yuan Tan, Singapore (SG); Sundaramoorthy Saravanan, Singapore (SG)

(21) Appl. No.: 14/354,923

(22) PCT Filed: Oct. 28, 2011
(86) PCT No.: PCT/SG2011/000383

§ 371 Date: Sep. 15, 2014

(51) Int. Cl. A61B 5/021; A61B 5/117; A61B 5/00

(52) U.S. Cl. CPC A61B 5/02141; A61B 5/0015; A61B 5/1172; A61B 5/7275
USPC 600/490

(57) ABSTRACT

A system for facilitating blood pressure management, the system comprising: a measuring device, a blood pressure cuff being connected to the measuring device and is adapted to wrap on a part of the body of the patient so that blood pressure measurement can be performed, an uploading application preinstalled in an information technology device, a server, the uploading application is adapted to transmit the blood pressure data from the measuring device to the server when the information technology device is connected online to the server via web services, wherein a doctor having connection to the server via web services can have access to the blood pressure data stored in the server and based on the blood pressure data, the doctor can provide feedback and consultation to the patient through communication means.

### CROSS REFERENCE TO RELATED APPLICATIONS

This is a U.S. national phase application claiming the benefit of and priority to PCT international application no. PCT/SG2011/000383, filed Oct. 28, 2011, currently pending, the entire disclosure of which is incorporated herein by reference.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

1. Field of the Invention

The present invention relates to a method and system to facilitate blood pressure management.

2. Background

Hypertension or high blood pressure is a condition in which the blood pressure in the arteries is chronically elevated. With every heart beat, the heart pumps blood through the arteries to the rest of the body. Blood pressure is the force of blood that is pushing up against the walls of the blood vessels. If the pressure is too high, the heart has to work harder to pump, and this could lead to organ damage and several illnesses such as heart attack, stroke, heart failure, aneurysm, or renal failure.

If a patient is diagnosed with hypertension, the doctor will take a medical history, examine him, check for signs of complications e.g. heart failure, do relevant blood and urine tests, do a chest X-ray and do an ECG (electrocardiogram).

The patient may also require to check his blood pressure at home to help him keep a closer watch on his blood pressure and manage his hypertension better. The patient may need to record down the blood pressure measurement results on a notebook and bring the results during the next medical appointment with the doctor. Such recording of the blood pressure data is not only error prone but also inconvenient too. There are some existing blood pressure monitors which can be linked with a computer. However, the patient can only store the blood pressure data locally into one computer, and has to print out the blood pressure data if he needs to show it to the doctor during the next medical appointment.

It is an object of the present invention to provide a method and system to facilitate blood pressure management which offers more convenience to the patient and also be less error prone.

Other objects and advantages of the present invention will become apparent from the following description, taken in connection with the accompanying drawings, wherein, by way of illustration and example, an embodiment of the present invention is disclosed.

### BRIEF SUMMARY OF THE INVENTION

In accordance with a first aspect of the invention, there is provided a system for facilitating blood pressure management, the system comprising: a measuring device, a blood pressure cuff being connected to the measuring device and is adapted to wrap on a part of the body of the patient so that blood pressure measurement can be performed, an uploading application preinstalled in an information technology device, a server, the uploading application is adapted to transmit the blood pressure data from the measuring device to the server when the information technology device is connected online to the server via web services, wherein a doctor having connection to the server via web services can have access to the blood pressure data stored in the server and based on the blood pressure data, the doctor can provide feedback and consultation to the patient through communication means.

In accordance with a second aspect of the invention, there is provided a method of measuring blood pressure of a patient for blood pressure management, the method comprising: providing a system according to claims 1 to 7, the measuring device providing a mode in the form of “select user” mode, displaying a list of registered users, identifying and selecting the patient's user account, displaying the personal information of the patient, providing a blood pressure cuff and connecting the blood pressure cuff to the measuring device, placing the blood pressure cuff onto a part of the body of the patient, performing inflation by the measuring device.

In accordance with a third aspect of the invention, there is provided a method of measuring blood pressure of a patient for blood pressure management, the method comprising: providing a system according to claims 1 to 7, the measuring device providing a mode in the form of “fingerprint” mode and providing a fingerprint sensor, placing a finger of the patient onto the fingerprint sensor, performing a validation process if the finger is detected, checking if the fingerprint has been previously registered, displaying the personal information of the patient if the fingerprint has been preciously registered, providing a blood pressure cuff and connecting the blood pressure cuff to the measuring device, placing the blood pressure cuff onto a part of the body of the patient, performing inflation by the measuring device.

In accordance with a fourth aspect of the invention, there is provided a method of measuring blood pressure of a patient for blood pressure management, the method comprising: providing a system according to claims 1 to 7, the measuring device providing a mode in the form of “immediate measurement” mode, providing a blood pressure cuff and connecting the blood pressure cuff to the measuring device, placing the blood pressure cuff onto a part of the body of the patient, performing inflation by the measuring device.

In accordance with a fifth aspect of the invention, there is provided a method of recording the blood pressure data of the patient after the blood measuring is completed, the method comprising: providing a system according to claims 1 to 7, performing inflation by the measuring device, providing a blood pressure cuff, detecting if the blood pressure cuff is properly wrapped on a part of the body of the patient, performing the measurement of the blood pressure of the patient if the blood pressure cuff is properly wrapped on a part of the body of the patient, detecting the heartbeat of the patient, flashing a heartbeat symbol at every heartbeat if the heartbeat of the patient is detected, obtaining blood pressure data, performing fast deflation, ending the fast deflation, determining if the measurement is valid, displaying the blood pressure data if the measurement is valid, prompting if the patient want to record and store the blood pressure data in the measuring device, recording and storing the blood pressure data in the measuring device if the patient indicate the same.

In accordance with a sixth aspect of the invention, there is provided a method of viewing the history records of the blood pressure of a patient, the method comprising: providing a system according to claims 1 to 7, the measuring device providing a mode in the form of “viewing past record” mode, selecting a mode in the form of “viewing past record” mode, displaying a list of registered users, identifying and selecting the patient's user account, displaying the personal information of the patient, selecting to view the blood pressure data in the form of history data table or trend graph, viewing the blood pressure data if measurement is taken within a specific time period.

In accordance with a seventh aspect of the invention, there is provided a method of viewing the history records of the blood pressure of a patient, the method comprising: providing a system according to claims 1 to 7, the measuring device providing a mode in the form of “fingerprint” mode, selecting a mode in the form of “fingerprint” mode, placing a finger of the patient onto the fingerprint sensor, performing a validation process if the finger is detected, checking if the fingerprint has been previously registered, displaying the personal information of the patient if the fingerprint has been preciously registered, selecting to view the blood pressure data in the form of history data table or trend graph, viewing the blood pressure data if measurement is taken within a specific time period.

In accordance with a eight aspect of the invention, there is provided a method of viewing the history records of the blood pressure of a patient, the method comprising: providing a system according to claims 1 to 7, the measuring device providing a mode in the form of “undefined user” mode, selecting a mode in the form of “undefined user” mode, displaying the blood pressure data in the form of history data table, viewing the blood pressure data if measurement is taken within a specific time period.

In accordance with a ninth aspect of the invention, there is provided a method of providing medical consultation by a doctor to a patient using the system according to claims 1 to 7, the method comprising: providing reminders with comments by the doctor, storing the reminders in the server, downloading the reminders from the server to the measuring device.

In accordance with a tenth aspect of the invention, there is provided a method of adding multimedia file to a blood pressure data using the system according to claims 1 to 7, the method comprising: selecting to add a multimedia file (in the form of text, voice or video that can be input through the invented device or when connect to mobile phone or connect to computer) to the blood pressure data stored in the measuring device, incorporating the multimedia file into the blood pressure data, uploading the blood pressure data with the multimedia file into the server, storing the blood pressure data with the multimedia file as a single file in the server, displaying identifier to show the presence of the multimedia file.

In accordance with a eleventh aspect of the invention, there is provided a method of selecting the size of the blood pressure cuff for blood pressure management, the method comprising: providing a system according to claims 1 to 7, providing a blood pressure cuff having an air tube connected thereto and a cuff size circuitry connected to the other end of the air tube, selecting the size of the blood pressure cuff during registration process, recording and storing the size of the blood pressure cuff under the patient's account, indicating the size of the blood pressure cuff by the cuff size circuitry, checking if the size of the blood pressure cuff tally with the record, carry on with the blood pressure measurement if the size of the blood pressure cuff tally with the record.

In accordance with a twelfth aspect of the invention, there is provided a method of detection of an artery in the hand of the patient during the process of blood pressure measurement, the method comprising: providing a system according to claims 1 to 7, providing the measuring device having an array of temperature sensors electrically connected to an amplifier and filter circuit which is in turn electrically connected to micro controller unit, the micro controller unit is controlled by the connector, scanning all temperatures and locating the artery by the micro controller unit, comparing the temperature closest to the blood pressure cuff and the highest temperature of the temperature sensors and thereby locating the error location, transmitting the error signal to the micro controller unit, prompting the patient to readjust the location of the blood pressure cuff on the arm.

In accordance with a thirteen aspect of the invention, there is provided a method of calibrating a measuring device in a calibration mode, the method comprising: providing a system according to claims 1 to 7, providing an algorithm preinstalled in the measuring device, the algorithm adapted for estimating the systolic blood pressure and diastolic blood pressure using an oscillometric method, adjusting diagnostic parameters by a doctor, wherein during a calibration mode, the method further comprise: listening for ausculatory sound on the patient using a stethoscope by the doctor and at the same time, performing inflation to increase the pressure of the blood pressure cuff, setting a new value of parameter by the doctor to overwrite the predefined parameter that detects the value for the systolic blood pressure, listening for ausculatory sound on the patient using a stethoscope by the doctor and at the same time, performing deflation to reduce the pressure of the blood pressure cuff, setting a new value of parameter by the doctor to overwrite the predefined parameter that detect the value for the diastolic blood pressure, providing inputs by the doctor by overwriting the systolic blood pressure and diastolic blood pressure detecting threshold parameters when the ausculatory sound is heard only effective to a particular patient the doctor is calibrating the measuring device for, storing the new value of the parameters in the patient's profile in the measuring device.

### BRIEF DESCRIPTION OF THE SEVERAL VIEWS OF THE DRAWING

By way of example/illustration only, an embodiment of the invention is described more fully hereinafter with reference to the accompanying drawings, in which:

FIG. 1 shows a graphical representation illustrates a system for facilitating blood pressure management.

FIG. 2 shows a flows chart illustrates the process of measuring the blood pressure of a patient.

FIG. 3 shows a flows chart illustrates the process of recording the blood pressure data of the patient after the measurement is completed.

FIG. 4 shows a flows chart illustrates the process of viewing the history records of the blood pressure data in the fingerprint mode.

FIG. 5 shows a graphical representation illustrates how the doctor provides medical consultation to the patient.

FIG. 6 shows a graphical representation illustrates the additional of a voice or video log file to the information technology device 610, such as a personal computer (PC).

FIG. 7 shows a graphical representation illustrates the selection of size of the blood pressure cuff.

FIG. 8 shows a graphical representation illustrates the detection of the artery in the hand during the process of the blood pressure measurement.

FIG. 9 shows a graphical representation illustrating a system and method of calibrating a measuring device in a calibration mode.

### DETAILED DESCRIPTION OF THE INVENTION

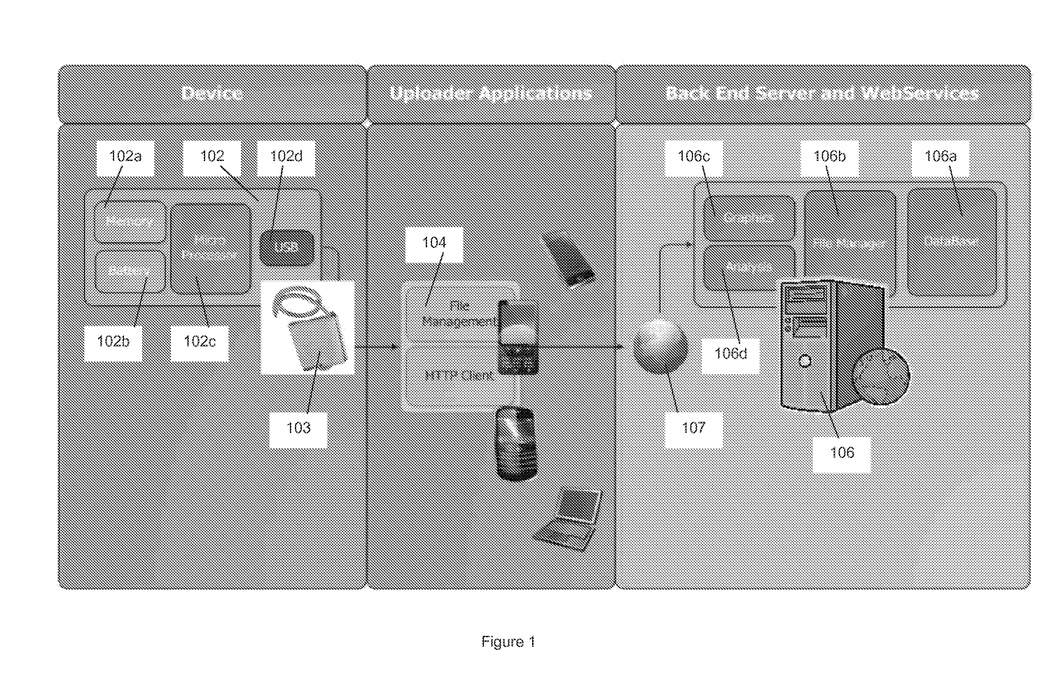

Referring now to FIG. 1, a graphical representation illustrates a system for facilitating blood pressure management. As shown, a measuring device 102 is provided to measure the blood pressure of the patient. After measuring the blood pressure, the patient uploads the blood pressure data into the server 106a by using an uploading application 104. The server 106 is also known as back end server and is connected online to a doctor via web services 107. The doctor is able to access the blood pressure data of the patient and thus able to provide feedback and consultation to the patient through email or SMS or other communication means.

The measuring device 102 includes a memory module 102a, a battery 102b, a micro processor 102c and a USB or other means of electronic input and output port 102d. The measuring device 102 is connected to a blood pressure cuff 103 via a tube and connector. The blood pressure cuff 103 is adapted to be placed smoothly and snugly around an upper arm, at roughly the same vertical height as the heart while the subject is seated with the arm supported. Other sites of placement depend on species, and may include the tongue, flipper, tail or teat. It is essential that the correct size of cuff is selected for the patient. Too small a cuff results in too high a pressure, while too large a cuff results in too low a pressure. The cuff is inflated until the artery is completely occluded. The device 102 will detect an oscillation as the cuff is inflated. The device 102 will predict the maximum pressure the cuff need to be inflated to, and release pressure gradually after the maximum pressure is achieved. The pressure at which this sound began is noted and recorded as the systolic blood pressure. The cuff pressure is further released until the sound can no longer be heard. This is recorded as the diastolic blood pressure, which will be referred to as blood pressure data in this patent application. The blood pressure data will be recorded and stored in the memory module 102a of the measuring device 102.

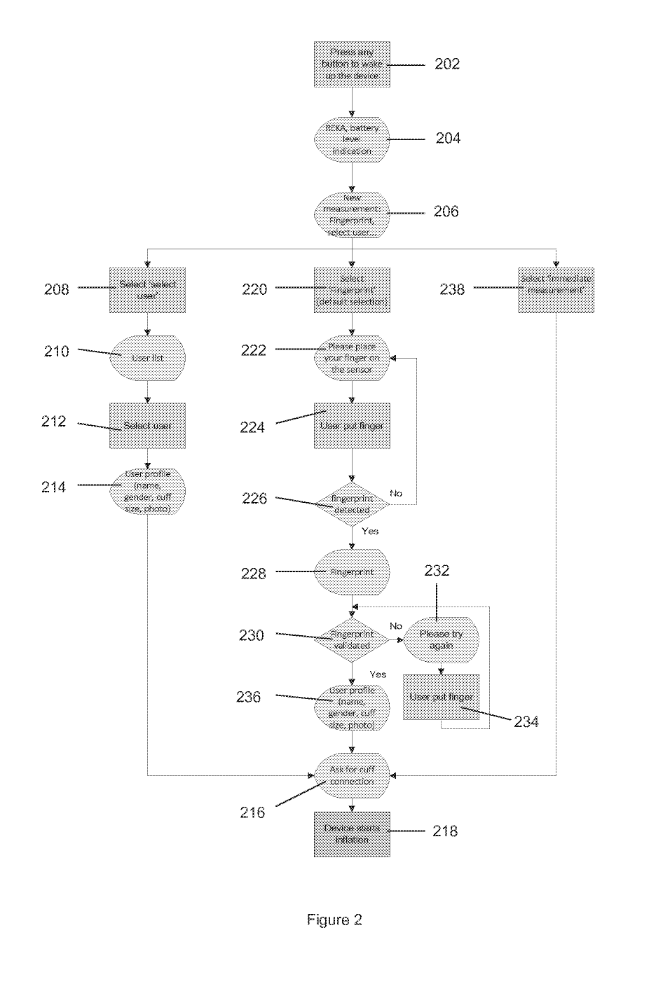

The measuring device 102 further includes a finger sensor (not shown). The finger sensor is adapted to ensure integrity, accuracy and security of the blood pressure data recorded if the “fingerprint mode” is selected. Other modes of operation will be discussed more in details later in FIG. 2. In the “fingerprint mode, before using the measuring device, the patient would places his finger on the finger sensor which will then perform an authentication test or identification test. If the patient is a registered user, the finger sensor would allow the patient to access to the functions of the measuring device 102 and also the history records of the blood pressure data of the patient. Thus, the authentication test or identification test performed by the finger sensors ensure that no authorised person can access to the function of the measuring device and also to the history records of the blood pressure data of the patient.

Since a patient has to first register with the measuring device 102 in order to use it in the “fingerprint mode”, the measuring device 102 also further includes a configuration software application adapted to allow the patient to register for a new account. The new account may include personalised information of the patient, such as blood pressure cuff size, maximum inflation pressure, blood pressure alarm value, register fingerprint, photo, personal profile and reminders. Once a new account is registered, the patient is able to use the measuring device 102. Every subsequent future usages of the measuring device in the “fingerprint mode” would require the patient to log in via the finger sensor.

The finger print used to identify the patient profile combined with medical device can be generalised to use in blood glucose, pulse oximetry, cholesterol and ECG devices.

An uploading application 104 is preinstalled in a typical information technological (IT) device such as a smart phone, a hand phone or a laptop. The uploading application 104 includes file management and HTTP client services and is adapted to upload the blood pressure data from the memory module 102a into the server 106 when the IT device is connected online to the server via web services 107.

The server 106 includes a database 106a, a file manager 106b, a graphics interface 106c and an analysis software program 106d. The database 106a stores the blood pressure data. The file manager 106b organises the blood pressure data. The graphics interface 106c displaces the blood pressure data in a graphical format. The analysis software program 106d analyses the blood pressure data. The server 106 is also known as a back end server.

The server 106 is connected online to a doctor via web services 107. An example of a web services is rekahealth.com. The doctor is able to access the blood pressure data of the patient and thus able to provide feedback and consultation to the patient through email or SMS or other communication means.

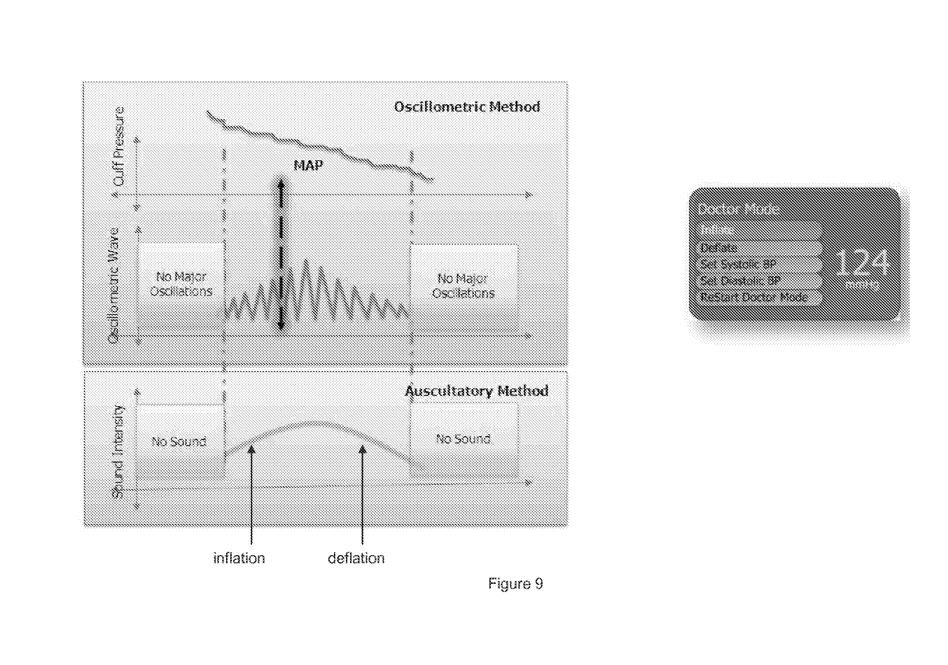

The measuring device 102 further includes a calibration mode which will be discussed more in detail in FIG. 9.

Referring now to FIG. 2, a flows chart illustrates the process of measuring the blood pressure of a patient.

The process starts at step 202 where the patient presses a button on the measuring device to activate it.

The power level of the battery will be shown in step 204.

The measuring device will indicate a new measurement to be performed at step 206. 3 modes of operation will be available to the patient. The first mode is the “select user” mode, the second mode is the “fingerprint mode” and the third mode is the “immediate measurement mode”.

If a patient selects the “select user” mode at step 208, the measuring device will display the list of registered user at step 210. The patient will then identify and select his user account at step 212. After selecting his user account, the measuring device will display the personal information of the patient, such as the blood pressure cuff size, maximum inflation pressure, blood pressure alarm value, register fingerprint, photo, personal profile and reminders at step 214. The patient will then connect the blood pressure cuff to the measuring device at step 216. Upon placing the blood pressure cuff on his arm or other parts of his body, the measuring device will start to perform inflation at step 218.

The patient can also select the “fingerprint mode”, which may be the default mode, at step 220. In this mode, the measuring device will prompt the patient to place his finger on a fingerprint sensor on the measuring sensor at step 222. When the patient places his finger on the fingerprint sensor at step 224, the fingerprint sensor will detect the finger print at step 226. If the fingerprint sensor is unable to detect the finger print, the finger print sensor will prompt the patient to place his finger again on the finger print sensor again at step 222. If the fingerprint sensor detects the finger print at step 228, the measuring sensor will perform validation process, which may also known as authentication test or identification test, at step 230. In this validation process, the measuring device will check if the finger print has been previously registered. If not, the finger print sensor will prompt the patient to try again at step 232 and the patient have to put the finger again on the finger sensor to validate one more time at step 234 and then the validation process restart at step 230. If the validation process is positive, the measuring device will display the personal information of the patient at step 236, such as the patient's name, the gender, the blood pressure cuff size and the patient's photo. The patient will then connect the blood pressure cuff to the measuring device at step 216. Upon placing the blood pressure cuff on his arm or other parts of his body, the measuring device will start to perform inflation at step 218.

The patient can also select the “immediate measurement” mode at step 238 which is more straightforward compared to the other two modes. In this mode, there is no need for any registration or any validation. There are also no history records of the blood pressure data of the patient. In this mode, the patient only needs to connect the blood pressure cuff to the measuring device at step 216. Upon placing the blood pressure cuff on his arm or other parts of his body, the measuring device will start to perform inflation at step 218.

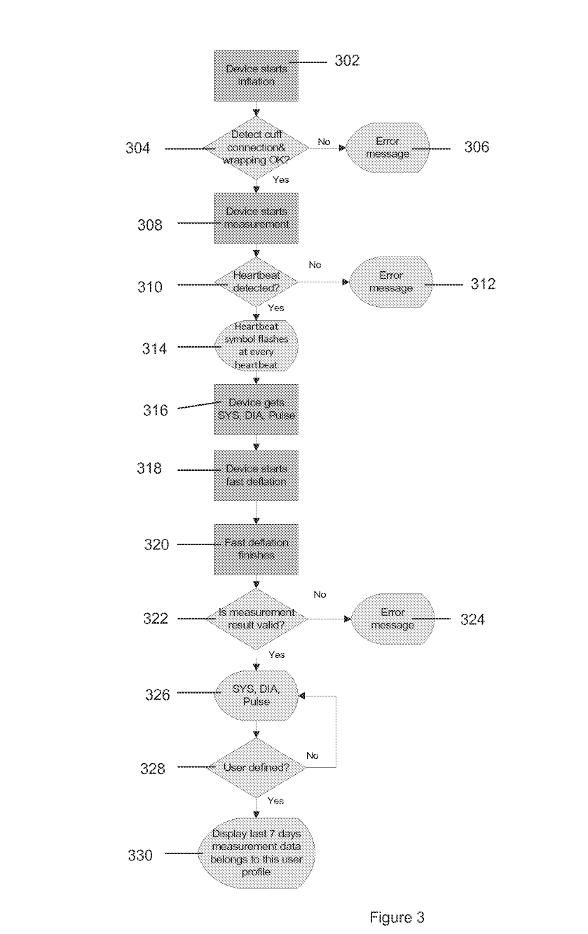

Referring now to FIG. 3, a flows chart illustrates the process of recording the blood pressure data of the patient after the measurement is completed.

The process starts at step 302 where the measuring device starts to perform inflation.

At step 304, the measuring device will detect if the blood pressure cuff is properly wrapped on the patient's arm or other parts of his body.

If not, the measuring device will prompt an error message at step 306 and the patient has to wrap the blood pressure cuff again on his arm or other parts of his body.

If the blood pressure cuff is properly wrapped on the patient's arm or other parts of his body, the measuring device will start to perform the measurement of the blood pressure of the patient at step 308.

At step 310, the measuring device will detect the heartbeat of the patient. If it is negative, the measuring device will prompt an error message at step 312. If it is positive, the measuring will display a heartbeat symbol flashing at every heartbeat at step 314.

At step 316, the measuring device obtains blood pressure data in the form of SYS, DIA, pulse, MAP, recorded time/date and device serial number information.

At step 318, the measuring device will perform fast deflation. The fast deflation process will end at step 320.

The measuring device will determine if the measurement is valid at step 322. If it is negative, the measuring will prompt an error message at step 324. If it is positive, the measuring device will display the blood pressure data of the patient at step 318. The displayed blood pressure data includes SYS, DIA and pulse.

The measuring device will then prompt if the patient want to record and store the blood pressure data in the measuring device at step 328. If not, the measuring device will simply display the blood pressure data of the patient. If so, the measuring device will record and store the blood pressure data of the patient, which can be retrievable up to previous 7 records, at step 330.

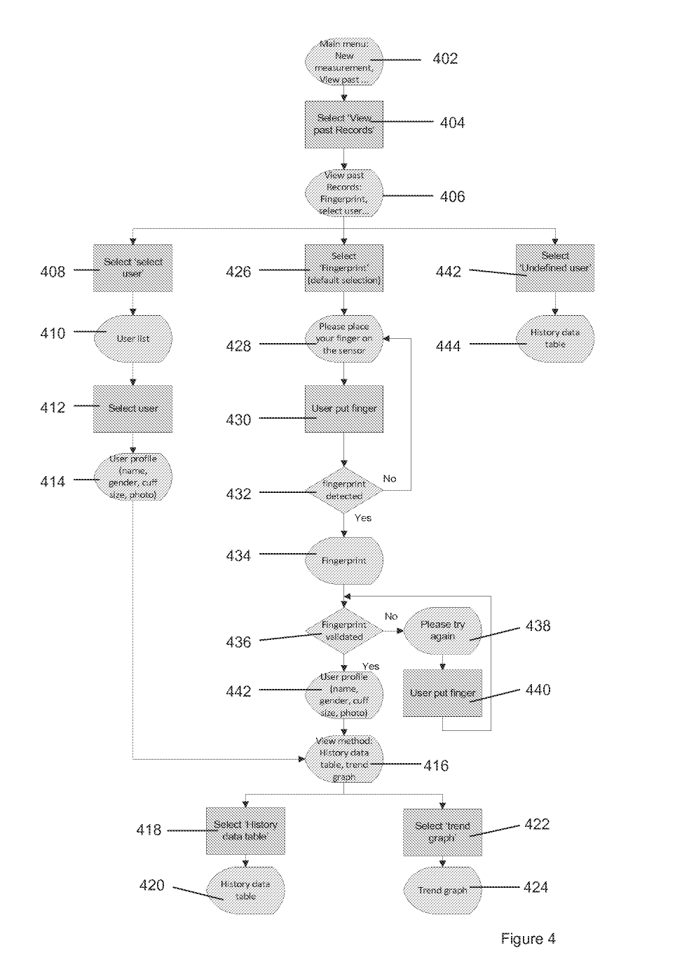

Referring now to FIG. 4, a flows chart illustrates the process of viewing the history records of the blood pressure data in the fingerprint mode.

The viewing procedure starts at step 402 where the measuring device display the main menu where the patient can choose to view the history records of his blood pressure data.

At step 404, the patient will select a mode in the main menu to view the past records of his blood pressure data.

After selecting the “viewing past records” mode, the measuring device will display 3 modes in which the patient can access to the past records of his blood pressure data at step 406.

If the first mode “select user” is selected at step 408, the measuring device will display a list of registered users at step 410. The patient will then identify and select his user account at step 412. After selecting his user account, the measuring device will display the personal information of the patient, such as the blood pressure cuff size, maximum inflation pressure, blood pressure alarm value, register fingerprint, photo, personal profile and reminders at step 414. The patient will then be able to view the blood pressure data which may in the form of history data, table and trend graph at step 416. If the patient wants to view the blood pressure data in the form of history data table, the patient can select the same at step 418 and the measuring device will display the same at step 420. If the patient wants to view the blood pressure data in the form of trend graph, the patient can select the same at step 422 and the measuring device will display the same at step 424. The trend graph can be presented for a specific interest group of data taken for a period of time (in weeks or months). The trend graph can be viewed according to measurements taken within a specific time period, such as morning (6 am to 10 am).

The second mode “fingerprint”, which may be the default mode, is selected at step 426. In this mode, the measuring device will prompt the patient to place his finger on a fingerprint sensor on the measuring sensor at step 428. When the patient places his finger on the fingerprint sensor at step 430, the fingerprint sensor will detect the finger print at step 432. If the fingerprint sensor is unable to detect the finger print, the finger print sensor will prompt the patient to place his finger again on the finger print sensor again at step 428. If the fingerprint sensor detects the finger print at step 434, the measuring sensor will perform validation process, which may also known as authentication test or identification test, at step 436. In this validation process, the measuring device will check if the finger print has been previously registered. If not, the finger print sensor will prompt the patient to try again at step 438 and the patient have to put the finger again on the finger sensor to validate one more time at step 440 and then the validation process restart at step 436. If the validation process is positive, the measuring device will display the personal information of the patient at step 442, such as the patient's name, the gender, the blood pressure cuff size and the patient's photo.

The patient will then be able to view the blood pressure data which may in the form of history data, table and trend graph at step 416. If the patient wants to view the blood pressure data in the form of history data table, the patient can select the same at step 418 and the measuring device will display the same at step 420. If the patient wants to view the blood pressure data in the form of trend graph, the patient can select the same at step 422 and the measuring device will display the same at step 424. The trend graph can be presented for a specific interest group of data taken for a period of time (in weeks or months). The trend graph can be viewed according to measurements taken within a specific time period, such as morning (6 am to 10 am).

The third mode “undefined user” is selected at step 442. In this mode, one can view the blood pressure data in the form of history data table of all registered users. The history data table can be presented for a specific interest group of data taken for a period of time (in weeks or months). The history data table can be viewed according to measurements taken within a specific time period, such as morning (6 am to 10 am).

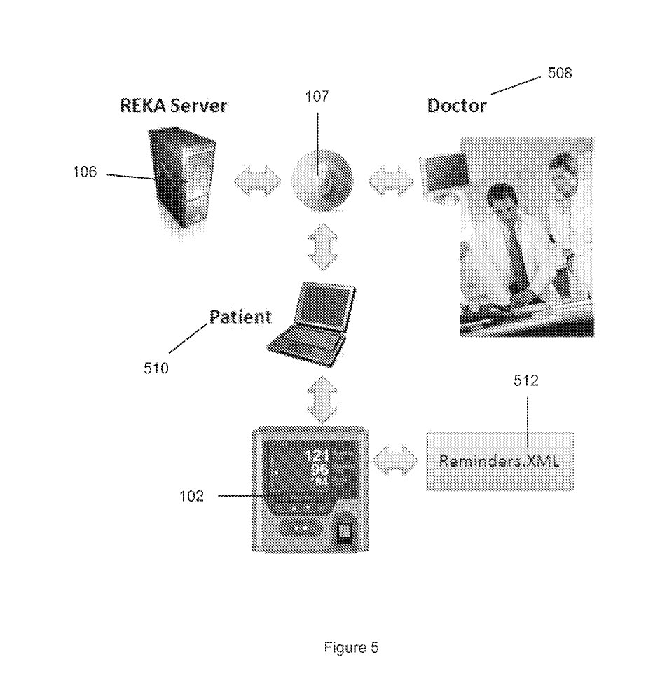

Referring now to FIG. 5, a graphical representation illustrates how the doctor provides medical consultation to the patient.

As discussed earlier in FIG. 1, the server 106 includes a database 106a, a file manager 106b, a graphics interface 106c and an analysis software program 106d. The database 106a stores the blood pressure data. The file manager 106b organises the blood pressure data. The graphics interface 106c displaces the blood pressure data in a graphical format. The analysis software program 106d analyses the blood pressure data. The server 106 is also known as a back end server.

The server 106 is connected online to a doctor 508 via web services 107. An example of a web services is rekahealth.com. The doctor is able to access the blood pressure data of the patient 510. The doctor may want to monitor the patient's blood pressure at a particular scenario or time. The doctor can also add new reminders with some comments. Such reminder is stored in the server 106 under the patient's account.

When the patient connects the measuring device 102 to the server 106 via web services 107, the patient uploads the blood pressure data into the server 106 by using an uploading application 104. The unloading application 104 is preinstalled in a typical information technological (IT) device such as a smart phone, a hand phone or a laptop. Thereafter the reminders which are stored in the server 106 will be downloaded to the measuring device 102 by means of the unloading application 104. The reminders 512 will comprise of comments from the doctor 508.

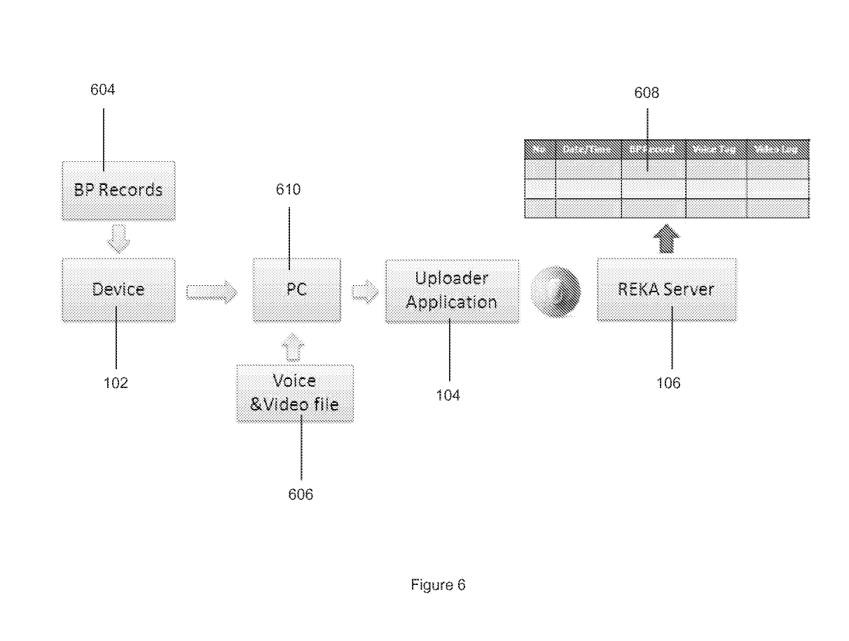

Referring now to FIG. 6, a graphical representation illustrates the additional of a voice or video log file to the information technology device 610, such as a personal computer (PC).

The blood pressure data 604 is recorded and stored in the measuring device 102. The patient then uploads the blood pressure data into the server 106 by using an uploading application 104. The unloading application 104 is preinstalled in a typical information technological (IT) device 610 such as a smart phone, a hand phone or a laptop.

The unloading application 104 may also prompt the patient if he wants to add a multimedia file, such as a voice or video log file 606. Other multimedia files may be in the form of text, voice or video that can be input through the invented device or when connect to mobile phone or connect to computer. If the patient adds the multimedia file, the blood pressure data 604 will incorporate the multimedia file 606 which will be uploaded into the server 106 by using an uploading application 104. The blood pressure data 606 with the corresponding multimedia file 606 will be stored as a single file 608 in the server 106. Identifier, such as special icons, would be displayed to show the presence of the multimedia file 606. If the multimedia file is present, the doctor will read the text file, listen to the voice log or play the video file in the multimedia file and the doctor will provide with the patient with additional information for diagnosis.

Combining the multimedia file 606 with the patient health records using measuring device 102 can be generalised to use in blood glucose, pulse oximetry, cholesterol and ECG devices.

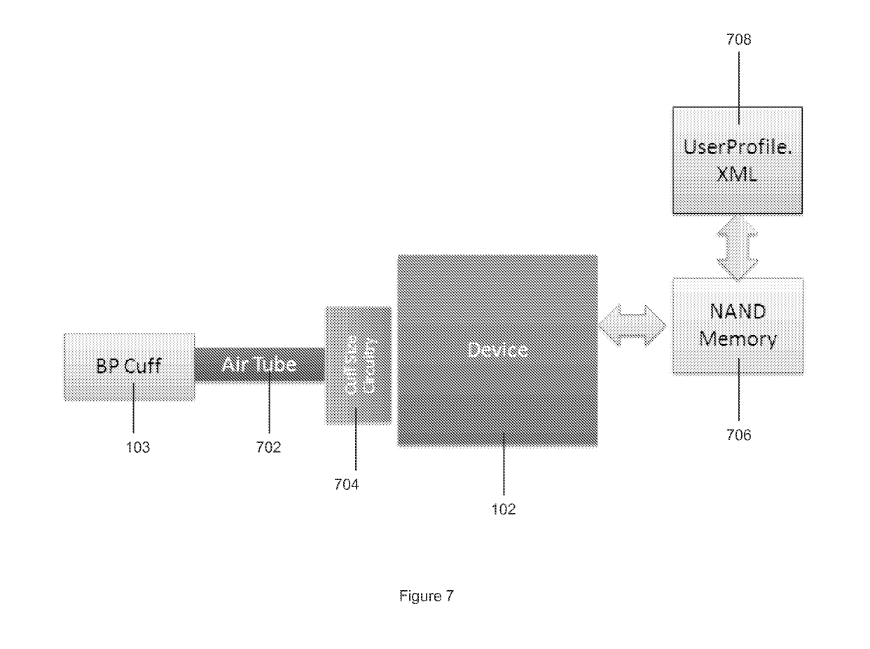

Referring now to FIG. 7, a graphical representation illustrates the selection of size of the blood pressure cuff.

During the initial registration process, the patient is required to select the size of his blood pressure cuff, amongst others. The size of his blood pressure cuff will be recorded and stored in the measuring device under the patient's account. In particular, the measuring device is provided with a memory unit in the form of NAND 706.The memory unit may also include other suitable memory units, beside NAND. The size of the blood pressure cuff will be stored in the file format 708 recorded and stored in the NAND706 in this embodiment.

The blood pressure cuff 103 includes an air tube 702 connected thereto and a cuff size circuitry 704 connected at the other end of the air tube 702. During measurement of the blood pressure, the circuitry 704 will indicate the size of the blood pressure cuff of the patient. If the size of the blood pressure cuff does not tally with the record stored in the NAND 706, the measuring device 102 will alert the patient. Otherwise, the patient can carry on with the measurement.

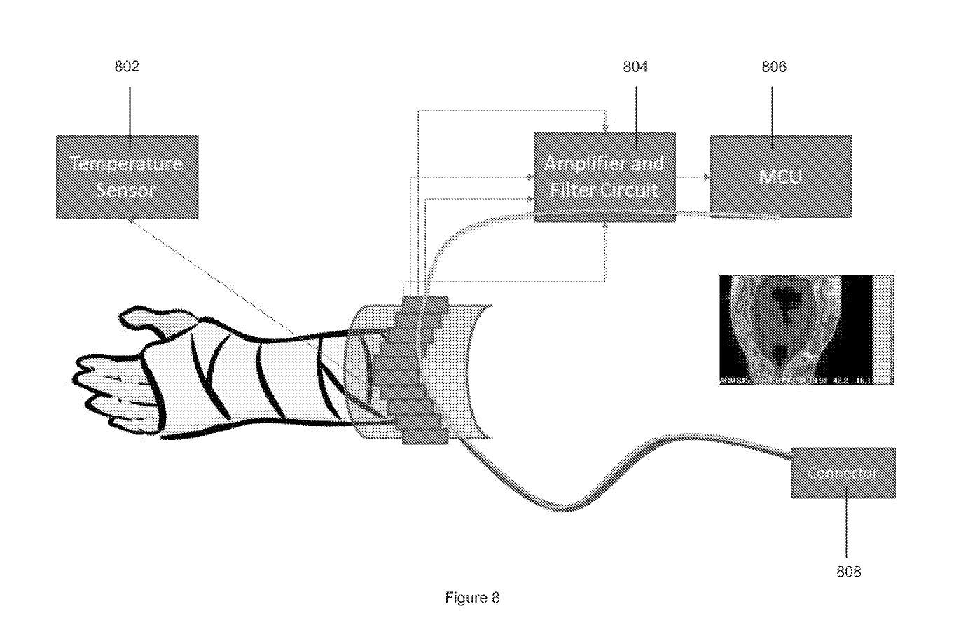

Referring now to FIG. 8, a graphical representation illustrates the detection of the artery in the hand during the process of the blood pressure measurement.

Brachial artery is the major vein supplying the blood to the forearm. The temperature on the blood vessels will be higher than the flesh of the skin.

The measuring device includes an array of temperature sensors 802 adapted to wrap around the arm of the patient, or other parts of the body. The temperature sensors 802 are electrically connected to an amplifier and filter circuit 804 which is in turn electrically connected to micro controller unit (MCU) 806. The micro controller unit 806 is controlled by the connector 808.

The micro controller unit 806 is adapted to scan all temperature values and locate the brachial artery. By comparing the temperature closest to the blood pressure cuff and the highest temperature of the temperature sensors 802, the micro controller unit 806 is able to locate the error location.

The error signal will be transmitted to the micro controller unit 806 which will prompt the patient to readjust the location of the blood pressure cuff on the arm.

Referring now to FIG. 9, a graphical representation illustrates a system and method of calibrating a measuring device in a calibration mode.

The measuring device is provided with a preinstalled algorithm for estimating the systolic blood pressure and diastolic blood pressure using an oscillometric method. The diagnostic parameters are adjusted by the doctor. The diagnostic parameters are the predefined threshold judging the systolic blood pressure and diastolic blood pressure values during a measurement using oscillometric method.

During the calibrating mode, the doctor listen for ausculatory sound on the patient using a stethoscope and at the same time perform inflation to increase the pressure of the blood pressure cuff. When listening to the ausculatory sound, the doctor can set a new value of parameter to overwrite the predefined parameter that detects the value for the systolic blood pressure.

The doctor will then perform deflation on the measuring device to reduce the pressure of the blood pressure cuff. When listening to the ausculatory sound, the doctor can set a new value of parameter to overwrite the predefined parameter that detects the value for diastolic blood pressure.

The doctor then provide inputs by overwriting the systolic blood pressure and diastolic blood pressure detecting threshold parameters when the ausculatory sound is heard only effective to a particular patient the doctor is calibrating the measuring device for. The new value of the parameters as a result of calibration is stored in the patient profile of the measuring device. This calibration method will personalize or customize the parameters for a specific patient that will greatly reduce the measurement error.

While the invention has been particularly shown and described with respect to a preferred embodiment thereof, it will be understood by those in the art that various changes in form and details can be made therein without departing from the spirit and scope of the invention.

1. A system for facilitating blood pressure management, the system comprising:
a measuring device,
a blood pressure cuff being connected to the measuring device and is adapted to wrap on a part of the body of the patient so that blood pressure measurement can be performed,
an uploading application preinstalled in an information technology device,
a server,
the uploading application is adapted to transmit the blood pressure data from the measuring device to the server when the information technology device is connected online to the server via web services,
wherein a doctor having connection to the server via web services can have access to the blood pressure data stored in the server and based on the blood pressure data, the doctor can provide feedback and consultation to the patient through communication means.

2. The system according to claim 1, wherein the measuring device includes a memory module, a battery, a micro processor and a USB port.

3. The system according to claim 2, wherein the measuring device further includes a finger sensor.

4. The system according to claim 3, wherein the measuring device further includes a configuration software.

5. The system according to claim 4, wherein the uploading application includes file management and HTTP client services.

6. The system according to claim 5, wherein the server includes a database, a file manager, a graphics interface and an analysis software program.

7. The system according to claim 6, wherein the measuring device further includes a calibration mode for the doctor to adjust a blood pressure threshold for a patient.

8. A method of measuring blood pressure of a patient for blood pressure management, the method comprising:
providing a system including a measuring device, the measuring device providing a mode in the form of a “select user” mode,
displaying a list of registered users,
identifying and selecting the patient's user account,
displaying the personal information of the patient,
providing a blood pressure cuff and connecting the blood pressure cuff to the measuring device,
placing the blood pressure cuff onto a part of the body of the patient, and
performing inflation by the measuring device.

9. The method of claim 8, further comprising:
the measuring device providing a mode in the form of a “fingerprint” mode, and providing a fingerprint sensor,
placing a finger of the patient onto the fingerprint sensor,
performing a validation process if the finger is detected,
checking if the fingerprint has been previously registered,
displaying the personal information of the patient if the fingerprint has been preciously registered,
providing a blood pressure cuff and connecting the blood pressure cuff to the measuring device,
placing the blood pressure cuff onto a part of the body of the patient, and
performing inflation by the measuring device.

10. The method of claim 9, further comprising:
the measuring device providing a mode in the form of “immediate measurement” mode.

11. The method of claim 10, further comprising:
detecting if the blood pressure cuff is properly wrapped on a part of the body of the patient,
performing the measurement of the blood pressure of the patient if the blood pressure cuff is properly wrapped on a part of the body of the patient,
detecting the heartbeat of the patient,
flashing a heartbeat symbol at every heartbeat if the heartbeat of the patient is detected,
obtaining blood pressure data,
performing fast deflation,
ending the fast deflation,
determining if the measurement is valid,
displaying the blood pressure data if the measurement is valid,
prompting if the patient want to record and store the blood pressure data in the measuring device, and
recording and storing the blood pressure data in the measuring device if the patient indicate the same.

12. The method of claim 11, further comprising:
the measuring device providing a mode in the form of a “viewing past record” mode,
selecting a mode in the form of “viewing past record” mode,
displaying a list of registered users,
identifying and selecting the patient's user account,
displaying the personal information of the patient,
selecting to view the blood pressure data in the form of history data table or trend graph, and
viewing the blood pressure data if measurement is taken within a specific time period.

13. The method of claim 12, further comprising:
the measuring device providing a mode in the form of “fingerprint” mode,
selecting a mode in the form of the “fingerprint” mode,
placing a finger of the patient onto the fingerprint sensor,
performing a validation process if the finger is detected,
checking if the fingerprint has been previously registered,
displaying the personal information of the patient if the fingerprint has been preciously registered,
selecting to view the blood pressure data in the form of history data table or trend graph, and
viewing the blood pressure data if measurement is taken within a specific time period.

14. The method of claim 13, further comprising:
the measuring device providing a mode in the form of a “undefined user” mode,
selecting a mode in the form of “undefined user” mode,
displaying the blood pressure data in the form of history data table, and
viewing the blood pressure data if measurement is taken within a specific time period.

15. The method of claim 14, further comprising:
providing reminders with comments by the doctor,
storing the reminders in the server, and
downloading the reminders from the server to the measuring device.

16. The method of claim 15, further comprising:
selecting to add a multimedia file to the blood pressure data stored in the measuring device,
incorporating the multimedia file into the blood pressure data,
uploading the blood pressure data with the multimedia file into the server,
storing the blood pressure data with the multimedia file as a single file in the server, and
displaying identifier to show the presence of the multimedia file.

17. The method of claim 16, further comprising:
providing the blood pressure cuff having an air tube connected thereto and a cuff size circuitry connected to the other end of the air tube,
selecting the size of the blood pressure cuff during registration process,
recording and storing the size of the blood pressure cuff under the patient's account,
indicating the size of the blood pressure cuff by the cuff size circuitry,
checking if the size of the blood pressure cuff tally with the record, and
carry on with the blood pressure measurement if the size of the blood pressure cuff tally with the record.

18. The method of claim 17, further comprising:
the measuring device having an array of temperature sensors electrically connected to an amplifier and filter circuit which is in turn electrically connected to micro controller unit, the micro controller unit is controlled by the connector,
scanning all temperatures and locating the artery by the micro controller unit,
comparing the temperature closest to the blood pressure cuff and the highest temperature of the temperature sensors and thereby locating the error location,
transmitting the error signal to the micro controller unit, and
prompting the patient to readjust the location of the blood pressure cuff on the arm.

19. The method of claim 18, further comprising:
providing an algorithm preinstalled in the measuring device, the algorithm adapted for estimating the systolic blood pressure and diastolic blood pressure using an oscillometric method,
adjusting diagnostic parameters by a doctor,
wherein during a calibration mode, the method further comprise:
listening for ausculatory sound on the patient using a stethoscope by the doctor and at the same time, performing inflation to increase the pressure of the blood pressure cuff,
setting a new value of parameter by the doctor to overwrite the predefined parameter that detects the value for the systolic blood pressure,
listening for ausculatory sound on the patient using a stethoscope by the doctor and at the same time, performing deflation to reduce the pressure of the blood pressure cuff,
setting a new value of parameter by the doctor to overwrite the predefined parameter that detect the value for the diastolic blood pressure,
providing inputs by the doctor by overwriting the systolic blood pressure and diastolic blood pressure detecting threshold parameters when the ausculatory sound is heard only effective to a particular patient the doctor is calibrating the measuring device for, and
storing the new value of the parameters in the patient's profile in the measuring device.
